(19) United States
(12) Patent Application Publication
(10) Pub. No.: US 2021/0210166 A1
(43) Pub. Date: Jul. 8, 2021

(54) IDENTIFYING CO-EVOLVING SITES AND SUBSTITUENT AMINO ACID RESIDUES

(71) Applicant: Samsung Electronics Co., Ltd., Suwon-si (KR)

(72) Inventors: Rajasekhara Reddy DUVVURU MUNI, Bangalore (IN); Tadi Venkata SIVA KUMAR, Bangalore (IN); Taeyong KIM, Daejeon (KR); Garima AGARWAL, Bangalore (IN); Priyadarshini Panemangalore PAI, Bangalore (IN)

(21) Appl. No.: 17/143,780

(22) Filed: Jan. 7, 2021

(30) Foreign Application Priority Data
Jan. 8, 2020 (IN) 202041000852
Jun. 5, 2020 (KR) 10-2020-0068606

(51) Int. Cl. G16B 30/10; G16B 40/00

(52) U.S. Cl. CPC G16B 30/10; G16B 40/00

(57) ABSTRACT

Embodiments herein disclose a method for identifying co-evolving sites and and at least one substituent amino acid residue. The method includes obtaining a current state of a protein and an ancestral state of the protein. Further, the method includes determining at least one amino acid substitution along with at least one co-evolving site associated with the protein based on the current state of the protein and the ancestral state of the protein. Further, the method includes assessing the at least one amino acid substitution as a function of a nucleotide substitution in the protein. Further, the method includes assessing at least one co-evolving site substitution based on the at least one assessed amino acid substitution. Further, the method includes identifying the co-evolving sites and at least one substituent amino acid residue.

### CROSS REFERENCE TO RELATED APPLICATION

This application claims the benefit of and priority to Indian Patent Application 202041000852 as filed on Jan, 8, 2020, and Korea Patent Application No.10-2020-0068606 as filed on Jun. 5, 2020, the disclosures of which are incorporated by reference herein in their entireties.

### BACKGROUND

### 1. Field

The present disclosure relates to protein engineering, and more specifically is related to a method and electronic device for identifying co-evolving sites and at least one substituent amino acid residue.

### 2. Description of the Related Art

Protein engineering is a labour intensive and time consuming task. Several computational resources are developed for identification of sites to engineer and amino acids to use for substitution at those sites with limited success. However, there is need for development of a procedure for detection of multiple sites that will affect a protein function and substitutions at those sites that will bring in rapid desirable changes in the protein function. The co-evolving sites and resulting amino acid substitutions in a protein family could be used to detect multiple sites from a sequence alone without the need for structure. Various methods are in use to detect co-evolving sites in the proteins. However, most successful methods deployed to date use either mutual information or entropy based measures to identify co-evolving positions. In general, the methods result in a large number of protein pairs and ranking them is a daunting task. The methods such as Direct Coupling Analysis (DCA) have used matrix algebra derived functions to score the site pairs and decouple the direct from indirect co-evolution with limited success. None of the existing methods can accurately detect the desired substitution at site/site pairs.

The existing methods can be used to detect co-evolving positions with structural and functional significance mostly dependent on amino acid substitutions retrieved from multiple sequence alignments. In existing methods, codon level nucleotide substitutions leading to different amino acid substitutions, i.e. radical and conservative, are not considered for co-evolving site and amino acid substitution prediction.

The existing methods can be used to detect the desired substitution at site pairs based on at least one of a correlation coefficient, an observed and expected patterns of data distribution, maximum likelihood, empirically derived contact probabilities, alignment perturbation, an information theory, and a machine learning approach as shown in the FIG. 1.

Thus, it is desired to address the above-mentioned disadvantages or other shortcomings or at least provide a useful alternative.

### SUMMARY

The principal object of the embodiments herein is to provide a method and electronic device for identifying co-evolving sites (i.e., functionally and structurally important co-evolving sites) and at least one substituent amino acid residue.

Another object of the embodiment herein is to obtain a current state of a protein and an ancestral state of the protein.

Another object of the embodiment herein is to determine at least one amino acid substitution along with at least one co-evolving site associated with the protein based on the current state of the protein and the ancestral state of the protein.

Another object of the embodiment herein is to assess the at least one amino acid substitution as a function of a nucleotide substitution in the protein.

Another object of the embodiment herein is to assess at least one co-evolving site substitution based on the at least one assessed amino acid substitution.

Another object of the embodiment herein is to identify the functionally and structurally important co-evolving sites and at least one substituent amino acid residue.

Another object of the embodiment herein is to rank co-evolved site pairs based on the at least one structurally and functionally important co-evolving site.

Another object of the embodiment herein is to assess the substitutions based on the at least one structurally and functionally important site.

Additional aspects will be set forth in part in the description which follows and, in part, will be apparent from the description, or may be learned by practice of the presented embodiments of the disclosure.

Accordingly, embodiments herein disclose a method for identifying co-evolving sites (e.g., functionally and structurally important co-evolving sites) and at least one substituent amino acid residue. The method includes obtaining, by an electronic device, a current state of a protein and an ancestral state of the protein. Further, the method includes determining, by the electronic device, at least one amino acid substitution along with at least one co-evolving site associated with the protein based on the current state of the protein and the ancestral state of the protein. Further, the method includes assessing, by the electronic device, the at least one amino acid substitution as a function of a nucleotide substitution in the protein. Further, the method includes assessing, by the electronic device, at least one co-evolving site substitution based on the at least one assessed amino acid substitution. Further, the method includes identifying, by the electronic device, the co-evolving sites and residues in the at least one amino acid sequence substitution based on the at least one assessed co-evolving site substitution.

In an embodiment, the method further includes ranking, by the electronic device, the at least one structurally and functionally important site in the at least one amino acid sequence substitution based on a nucleotide transition and a transversion propensity.

In an embodiment, the method further includes ranking, by the electronic device, co-evolved site pairs based on the at least one structurally and functionally important site in the at least one amino acid sequence substitution.

In an embodiment, the method further includes assessing, by the electronic device, another amino acid substitution based on the co-evolving sites.

In an embodiment, the at least one co-evolving site substitution is assessed by a type of nucleotide mutation resulting in co-evolving site substitution and assessing a selection pressure on each site.

In an embodiment, the at least one amino acid substitution is assessed as the function of the nucleotide substitution using a custom built amino acid substitution matrix, wherein the amino acid substitution matrix reflects a normalized score of count and type of nucleotide substitutions causing amino acid shift.

In an embodiment, the at least one structurally and functionally important site is ranked based on a weighted average rank of type of nucleotide substitution in the protein and a frequency of the at least one amino acid sequence with positional pair.

In an embodiment, assessing, by the electronic device, the at least one amino acid substitution as the function of the nucleotide substitution in the protein includes grouping the nucleotide substitution based on a nucleotide transition and a transversion propensity, wherein the nucleotide transition includes substitutions within purines and transversion propensity includes substitutions within pyrimidines, and assessing the at least one amino acid substitution as the function of the nucleotide substitution in the protein based on the nucleotide transition and the transversion propensity.

In an embodiment, obtaining the current state of the protein and the ancestral state of the protein includes compiling sequence homologs across taxa, filtering sequence homologs, building multiple sequence alignments, and obtaining the current state of the protein and the ancestral state of the protein based on the multiple sequence alignments.

Accordingly, embodiments herein disclose an electronic device for identifying co-evolving sites (e.g., functionally and structurally important co-evolving sites) and at least one substituent amino acid residue. The electronic device includes a processor coupled with a memory storing instructions. Upon execution of the instructions, the processor is configured to obtain a current state of the protein and an ancestral state of the protein. Further, the processor is configured to determine at least one amino acid substitution along with at least one co-evolving site associated with the protein based on the current state of the protein and the ancestral state of the protein. Further, the processor is configured to assess the at least one amino acid substitution as a function of a nucleotide substitution in the protein. Further, the processor is configured to assess at least one co-evolving site substitution based on the at least one assessed amino acid substitution. Further, the processor is configured to identify the co-evolving sites (functionally and structurally important co-evolving sites) and residue(s) in the at least one amino acid sequence substitution.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

This method is illustrated in the accompanying drawings, throughout which like reference letters indicate corresponding parts in the various figures. The embodiments herein will be better understood from the following description with reference to the drawings, in which:

FIG. 1 is an example scenario in which an electronic device identifies co-evolving sites (functionally and structurally important co-evolving sites) and residues in at least one amino acid sequence substitution according to a prior method;

FIG. 2 illustrates various hardware components of an electronic device for identifying functionally and structurally important co-evolving sites and at least one substituent amino acid residue, according to an embodiment as disclosed herein;

FIG. 3 illustrates various hardware components of a processor included in the electronic device for identifying functionally and structurally important co-evolving sites and at least one substituent amino acid residue, according to an embodiment as disclosed herein;

FIG. 4 is an example scenario in which site pair identification is depicted, according to an embodiment as disclosed herein;

FIG. 5 represents a genetic code which compiles information regarding all the possible codons in DNA that can code for an amino acid along with triple letter and single letter amino acid names, according to prior art;

FIG. 6 represents nucleotide substitution types, namely transitions and transversions, that result due to changes that occur in the DNA when a nucleotide gets substituted either within purines (Adenine and Guanine) or pyramidins (Cytosine and Thymine) or between purines and pyramidins, according to prior art;

FIG. 7 is an example representation for degeneracy in codons coding for Leucine (CTG, CTA, CTT, CTC, TTG, TTA) and the single nucleotide substitutions at various positions that lead to coding for different amino acids [CTG→CCG (PRO), CCG (Arg), CAG (Gly) changes in middle position], Changes in first position [CTG (Leu)→ATG (Met), GTG (Val)], according to prior art;

FIG. 8 illustrates an example of nucleotide substitutions that can cause amino acid change from Leucine to Proline, according to an embodiment as disclosed herein;

FIG. 9 illustrates an example of nucleotide substitutions that can cause amino acid change from Cyctein to Leucine, according to an embodiment as disclosed herein;

FIG. 10 is an example amino acid substitution matrix as a function of nucleotide transition substitution propensity, according to an embodiment as disclosed herein;

FIG. 11 is an example amino acid substitution matrix as a function of nucleotide transversion substitution propensity, according to an embodiment as disclosed herein; and

FIG. 12 is a flow chart illustrating a method for identifying functionally and structurally important co-evolving sites and at least one substituent amino acid residue, according to an embodiment as disclosed herein.

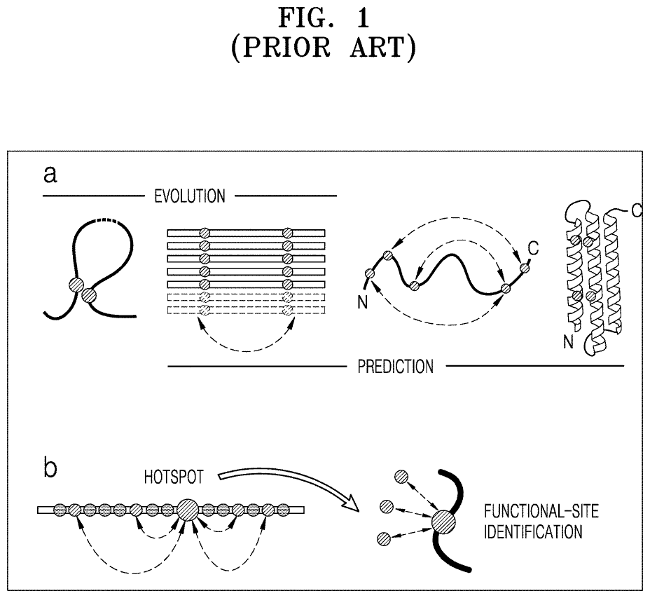

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

As is traditional in the field, embodiments may be described and illustrated in terms of blocks which carry out a described function or functions. These blocks, which may be referred to herein as units or modules or the like, are physically implemented by analog or digital circuits such as logic gates, integrated circuits, microprocessors, microcontrollers, memory circuits, passive electronic components, active electronic components, optical components, hardwired circuits, or the like, and may optionally be driven by firmware and software. The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the invention. Likewise, the blocks of the embodiments may be physically combined into more complex blocks without departing from the scope of the invention

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

Below are definition for certain terms used in the disclosure:

The functionally and structurally important co-evolving sites: A protein biological activity can be regulated by sites that are involved in ligand binding, tunnel lining resides, active site residues, binding site lining residues, etc. which are directly involved in ligand interactions. Though it cannot be generalized, often these sites are clustered in a protein sequence space. On the other hand, some sites in proteins tightly regulate the stability of three dimensional structure of the protein which in turn may determine the biological activity of the protein such as sites involved in intra and inter molecular contact, etc. These sites need not be in close proximity in sequence space.

Co-Evolving site substitution: The Co-Evolving sites need not carry the same substitution of amino acids, so identification of the substitution which is more impactful in the co-evolving pair is important.

Co-evolving site pair: When the electronic device analyses the protein for identification of co-evolving sites, it is possible that a site may be co-evolving with many other sites. But of all these, only some are only undergoing the required type of co-evolution. Only such things are considered as a pair. Or alternatively each of the combination will also form a co-evolving pair.

Accordingly, embodiments herein achieve a method for identifying co-evolving sites (functionally and structurally important co-evolving sites) and at least one substituent amino acid residue. The method includes obtaining, by an electronic device, a current state of a protein and an ancestral state of the protein. Further, the method includes determining, by the electronic device, at least one amino acid substitution along with at least one co-evolving site associated with the protein based on the current state of the protein and the ancestral state of the protein. Further, the method includes assessing, by the electronic device, the at least one amino acid substitution as a function of a nucleotide substitution in the protein. Further, the method includes assessing, by the electronic device, at least one co-evolving site substitution based on the at least one assessed amino acid substitution. Further, the method includes identifying, by the electronic device, the co-evolving sites and residues in the at least one amino acid sequence substitution.

Unlike conventional methods and systems, the proposed methods can be used to identify the functionally and structurally important co-evolving sites and at least one substituent amino acid residue without limitation on protein property and without depending on protein structure. The methods can be used to identify the functionally and structurally important co-evolving sites and at least one substituent amino acid residue in a cost and time effective manner. In the proposed methods, a method may utilize qualitative (transition versus transversion) and quantitative (number of each type) nucleotide substitutions leading to amino acid changes in protein of choice accounting for accurate selection pressure. In the proposed methods, flexibility to define current and ancestral states of the protein enables multiple rounds of protein optimization.

The proposed methods may utilize ancestral state construction of a given protein and identify substitutions along with co-evolving sites. The methods can be used to accurately predict sites and substitutions that will impact affinity and thermal stability and substrate specificity. The methods can be used to predict intra-atom contacts more accurately. By altering the ranking weights, a method can be used to accurately predict the functionally important sites in ˜8000 Conserved Domain Database (CDD) domains.

The methods can be used to identify co-evolving pairs excluding conserved amino acids and assess the substitutions based on type of nucleotide mutations. This results in substitution and assessment of selection pressure on each site in an effective manner. The methods can be used to build the amino acid substitution matrix reflecting count of type of nucleotide substitutions causing amino acid shift. The methods can be used to identify co-evolving sites using a consensus per site. The methods can be used to identify intra-atom contacts and substitutions of functional relevance.

The proposed methods consider both type of transversion and transition and number of mutation events at DNA level that could guide protein evolution from an ancestral state at each position and for position pair.

The proposed methods can be used to predict the structural contacts, site/sites and substituent amino acid prediction, and provide a thermal stability assessment. The proposed methods can be used to reduce turn-around time of enzyme engineering by limiting amino acid substitutions. The proposed methods can be used for any choice of the protein function. The protein function may be defined by the user.

The proposed methods can be used to identify an intra-molecular structural contact. The proposed methods may utilize multiple sequence alignment with representative homologues bearing minimum redundancy and maximum diversity with target amino acid sequence for filtering the homologs. The proposed methods can be used to identify co-evolving positional pairs, whose constituent amino acids are different than the ancestral state and computation of summary statistics. The proposed methods can be used to provide relative ranking of positional pairs using weighted average as required for identification of structural contacts identification, functional site identification.

The methods can be implemented, for example, but not limited to an enzyme engineering, receptor design and protein structural biology with applications in pharmaceutical, biochemical synthesis and degradation and other biotechnological industries.

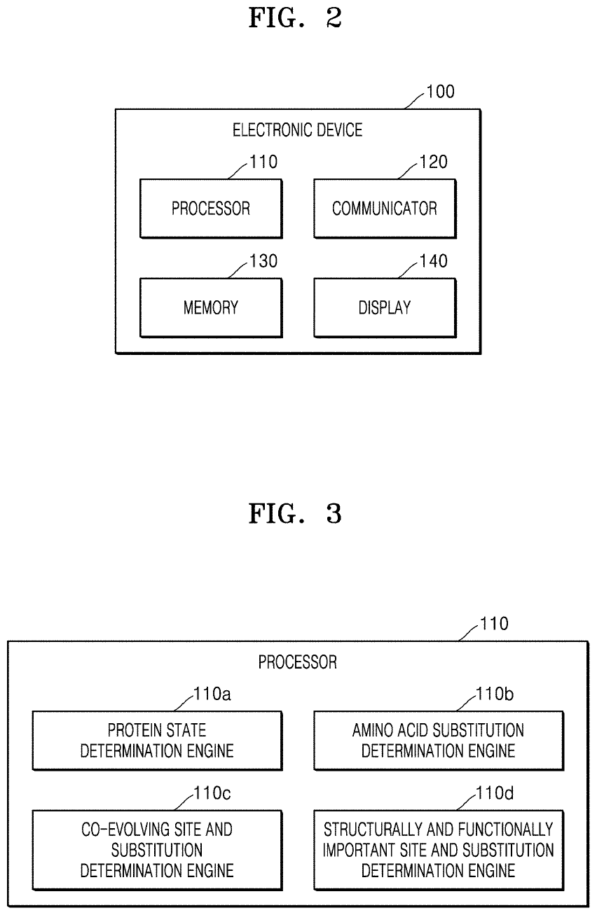

Referring now to the drawings, and more particularly to FIGS. 2 to 4 and 8 to 12, there are shown certain embodiments.

FIG. 2 illustrates various hardware components of an electronic device (100) for identifying functionally and structurally important co-evolving sites and at least one substituent amino acid residue, according to an embodiment as disclosed herein. The electronic device (100) can be, for example, but not limited to a cellular phone, a smart phone, a Personal Digital Assistant (PDA), a tablet computer, a laptop computer, an Internet of Things (IoT), a smart watch or the like.

In an embodiment, the electronic device (100) includes a processor (110), a communicator (120), a memory (130) and a display (140). The processor (110) is coupled with the communicator (120), the memory (130) and the display (140).

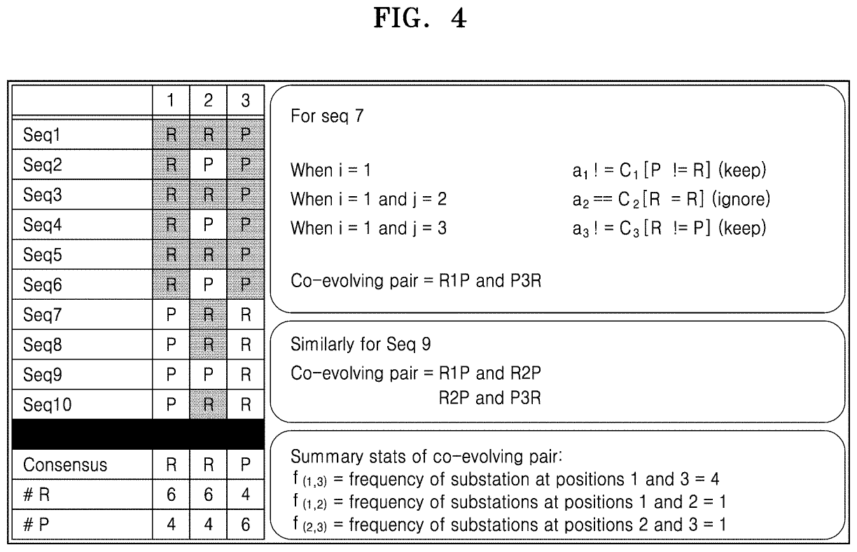

In an embodiment, the processor (110) is configured to obtain a current state of a protein and an ancestral state of the protein. In an embodiment, the current state of the protein and the ancestral state of the protein are obtained by compiling sequence homologs across taxa, filtering sequence homologs, building multiple sequence alignments, and obtaining the current state of the protein and the ancestral state of the protein based on the multiple sequence alignments as shown in FIG. 4. The ancestral state of the protein is computed based on the existing techniques (e.g., maximum parsimony techniques, maximum likelihood techniques, Bayesian inference techniques, consensus based techniques or the like).

Compiling sequence homologs across taxa: Target protein will be used to probe reference sequence databases using a homology search procedure such as BLAST.

Filtering sequence homologs: Sequence homologs obtained will be filtered based on:
1. Eliminating the sequences from different strains,
2. Truncated sequences (Sequence length less than the mean sequence length of all the proteins in data set), and
3. Sequences with less than 50% identity across protein length.

Building multiple sequence alignments: Filtered set of sequences will be subjected to multiple sequence alignment and the resulting alignment will be filtered for gaps in alignment.

Obtaining the current state of the protein and the ancestral state of the protein based on the multiple sequence alignments: Ancestral state of the protein can be computed by several ways such as consensus/Monte Carlo simulation/Bayesian statistics etc. Current state of the protein is the target protein under investigation in subsequent round of protein optimization.

Further, the processor (110) is configured to determine at least one amino acid substitution along with at least one co-evolving site associated with the protein based on the current state of the protein and the ancestral state of the protein. Further, the processor (110) is configured to assess the at least one amino acid substitution as a function of a nucleotide substitution in the protein. In an embodiment, the at least one amino acid substitution is assessed as the function of the nucleotide substitution in the protein using an amino acid substitution matrix, wherein the amino acid substitution matrix reflects count of type of nucleotide substitutions causing amino acid shift.

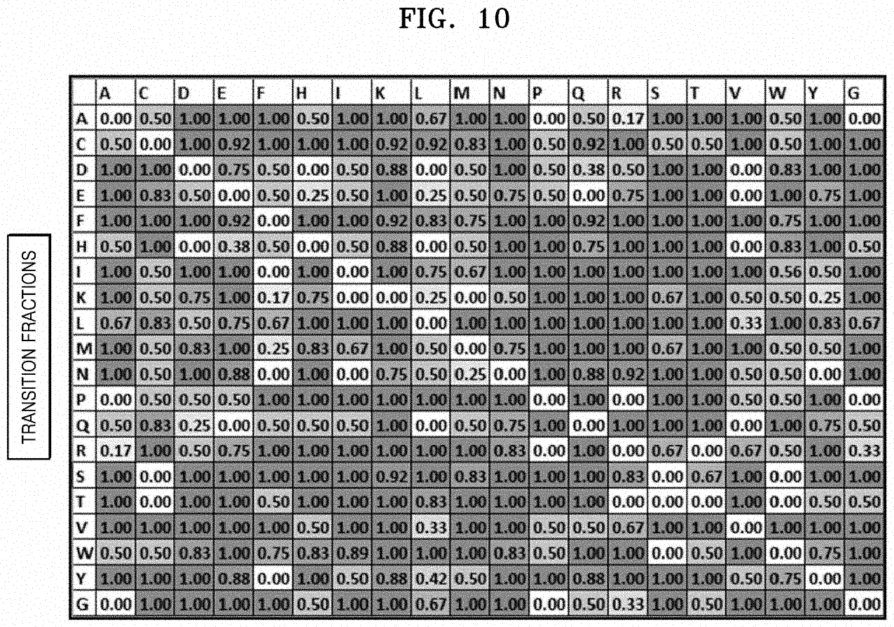

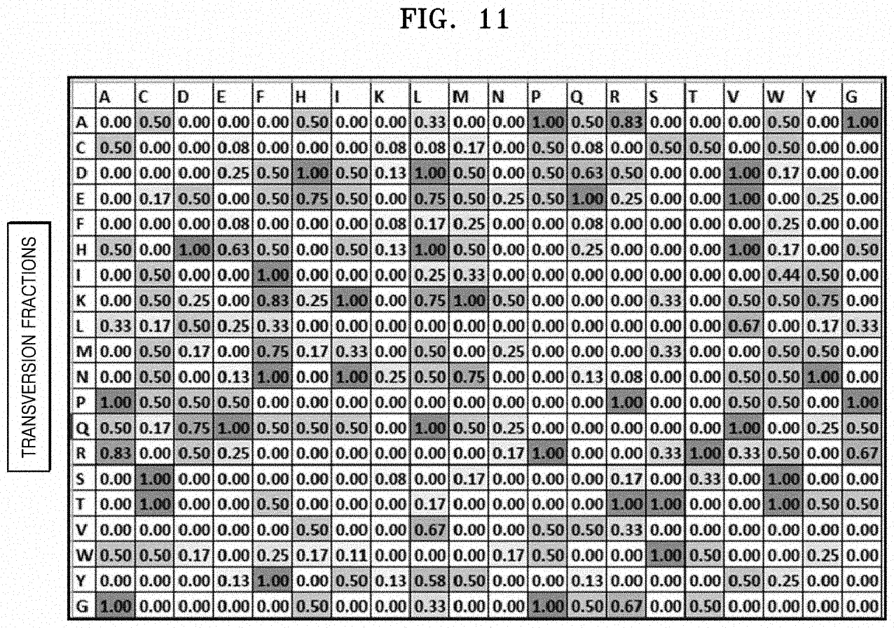

In an embodiment, the at least one amino acid substitution as the function of the nucleotide substitution in the protein is assessed by grouping the nucleotide substitution based on a nucleotide transition as shown in FIG. 10 and a transversion propensity as shown in FIG. 11, wherein the nucleotide transition includes substitutions within purines (i.e. Adenine and Guanine) and transversion propensity includes substitutions within pyrimidines (i.e., Cytosine and Thymine), and assessing the at least one amino acid substitution as the function of the nucleotide substitution in the protein based on the nucleotide transition and the transversion propensity.

The genetic code consists of triplets of nucleotides (Codon) and code for a particular type of amino acid. Due to degeneracy of a genetic code, multiple codons can code for the same amino acid. Due to this fact, different nucleotide substitutions (both number of substitutions, i.e. all the three positions of codons or two of the positions of the codon, or a single position of codon, and the type of substitutions, i.e. transition or transverison) could be tolerated. The processor (110) can be used to build an amino acid substitution matrix based on the number and type of substitution required for successful conversion of one amino acid to another based on number and type of nucleotides required.

In an embodiment, using the amino acid substitution matrix, the processor (110) computes a transition fraction at position Pᵢ:: tₛ(Pᵢ) and a transition fraction at position Pⱼ:: tₛ(Pⱼ).

In an embodiment, using amino acid substitution matrix, the processor (110) computes a transversion fraction at position Pᵢ:: tᵥ(Pᵢ) and a transversion fraction at position Pⱼ:: tᵥ(Pⱼ).

In an embodiment, using amino acid substitution matrix, the processor (110) computes a mean transition fraction for pair Pᵢ and Pⱼ::

$${μ}_{{ts}_{( ij )}} = \frac{{t}_{s} ( {P}_{i} ) + {t}_{s} ( {P}_{j} )}{2}$$

and a mean transversion fraction at position Pᵢ and Pⱼ::

$${μ}_{{tv}_{( ij )}} = \frac{{t}_{v} ( {P}_{i} ) + {t}_{v} ( {P}_{j} )}{2} .$$

Further, the processor (110) is configured to assess at least one co-evolving site substitution based on the at least one assessed amino acid substitution. In an embodiment, the at least one co-evolving site substitution is assessed by a type of nucleotide mutations resulting in co-evolving site substitution and assessing the selection pressure on each site.

Further, the processor (110) is configured to identify the functionally and structurally important co-evolving sites and residues in the at least one amino acid sequence substitution.

In an embodiment, the processor (110) is configured to rank the at least one structurally and functionally important site in the at least one amino acid sequence substitution based on the nucleotide transition and the transversion propensity. In an embodiment, the at least one structurally and functionally important site is ranked based on a weighted average rank of type of nucleotide substitution in the protein and a percentage of the at least one amino acid sequence with positional pair.

In an embodiment, the processor (110) is configured to rank co-evolved site pairs based on the at least one structurally and functionally important sites in the at least one amino acid sequence substitution.

In an embodiment, the processor (110) is configured to assess the substitutions based on the at least one structurally and functionally important site in the at least one amino acid sequence substitution.
Site pair rank for Pᵢ & Pⱼ is:

$${r}_{( i , j )} = \frac{{w}_{1} {f}_{( i , j )} + {w}_{2} {μ}_{t {s}_{( i , j )}} + {w}_{3} {μ}_{t {v}_{( i , j )}}}{{w}_{1} + {w}_{2} + {w}_{3}}$$

where f_{(i,j)}=Frequency of site pair (Pᵢ & Pⱼ)
μₜₛ_{_{(i,j)}}=Average of transitions per pair (Pᵢ & Pⱼ)
μₜᵥ_{_{(i,j)}}=Average of transversions per pair (Pᵢ & Pⱼ)
w₁=Weight for frequency of pair
w₂=Weight for pairs under going transversions
w₃=Weight for pairs under going transitions

Weight adjustment for target application:
1. A pair that had appeared in a maximum number of sequences represents a substitution pair is subjected to purifying selection
2. A pair that had undergone transversions will have caused drastic changes in protein at sites
3. A pair that had undergone transitions is required for functional conservation.

The processor (110) is configured to execute instructions stored in the memory (130) and to perform various processes. The communicator (120) is configured for communicating internally between internal hardware components and with external devices via one or more networks.

The memory (130) also stores instructions to be executed by the processor (110). The memory (130) may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory (130) may, in some examples, be considered a non-transitory storage medium. The term “non-transitory” may indicate that the storage medium is not embodied in a carrier wave or a propagated signal. However, the term “non-transitory” should not be interpreted that the memory (130) is non-movable. In some examples, the memory (130) can be configured to store larger amounts of information than the memory. In certain examples, a non-transitory storage medium may store data that can, over time, change (e.g., in Random Access Memory (RAM) or cache).

Although FIG. 2 shows various hardware components of the electronic device (100) it is to be understood that other embodiments are not limited thereon. In other embodiments, the electronic device (100) may include less or more number of components. Further, the labels or names of the components are used only for illustrative purpose and does not limit the scope of the invention. One or more components can be combined together to perform same or substantially similar function to identifying functionally and structurally important co-evolving sites and at least one substituent amino acid residues.

FIG. 3 illustrates various hardware components of the processor (110) included in the electronic device (100) for identifying functionally and structurally important co-evolving sites and at least one substituent amino acid residue in at least one amino acid sequence substitution, according to an embodiment as disclosed herein. In an embodiment, the processor (110) includes a protein state determination engine (110a), an amino acid substitution determination engine (110b), a co-evolving site and substitution determination engine (110c) and a structurally and functionally important site and substitution determination engine (110d).

The protein state determination engine (110a) is configured to obtain the current state of the protein and the ancestral state of the protein. The amino acid substitution determination engine (110b) is configured to determine the at least one amino acid substitution along with the at least one co-evolving site associated with the protein based on the current state of the protein and the ancestral state of the protein. Further, the amino acid substitution determination engine (110b) is configured to assess the at least one amino acid substitution as the function of the nucleotide substitution in the protein. Further, co-evolving site and substitution determination engine (110c) is configured to assess the at least one co-evolving site substitution based on the at least one assessed amino acid substitution. The structurally and functionally important site and substitution determination engine (110d) is configured to identify the functionally and structurally important co-evolving sites and residues in the at least one amino acid sequence substitution.

Although FIG. 3 shows various hardware components of the processor (110) it is to be understood that other embodiments are not limited thereon. In other embodiments, the processor (110) may include less or more number of components. Further, the labels or names of the components are used only for illustrative purpose and does not limit the scope of the invention. One or more components can be combined together to perform same or substantially similarfunction to identifying functionally and structurally important co-evolving sites and residues in at least one amino acid sequence substitution.

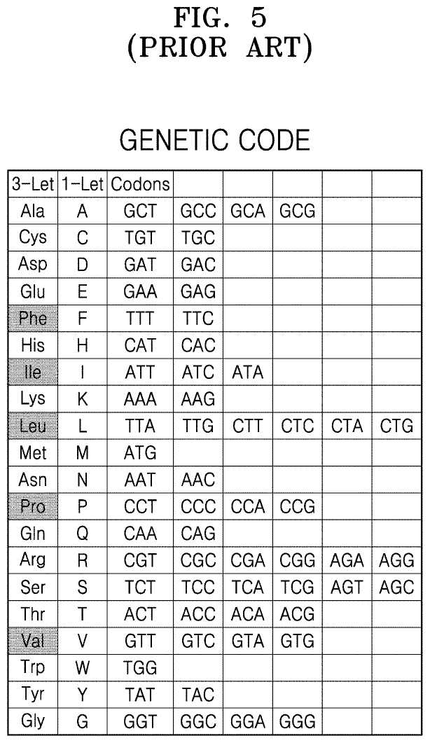

FIG. 5 represents a genetic code which compiles information regarding all the possible codons in DNA that can code for an amino acid along with triple letter and single letter amino acid names, according to prior art.

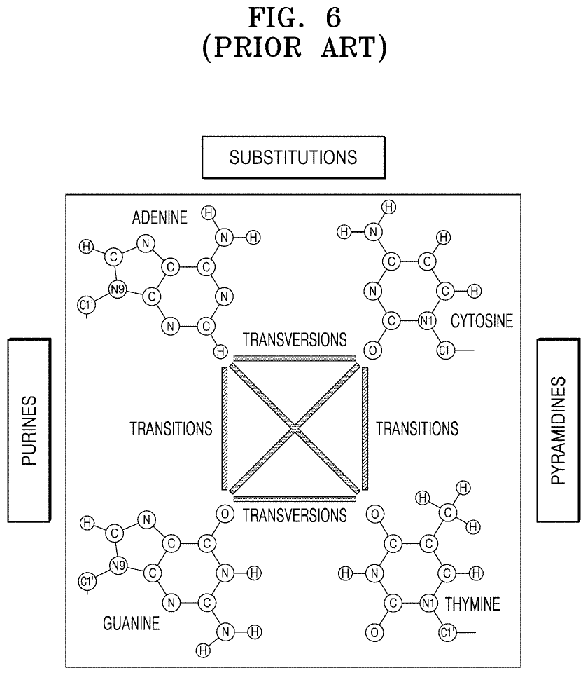

FIG. 6 represents nucleotide substitution types namely transitions and transversions that results due to changes that occur in the DNA when a nucleotide gets substituted either within purines (Adenine and Guanine) or pyramidins (Cytosine and Thymine) or between purines and pyramidins, according to prior art.

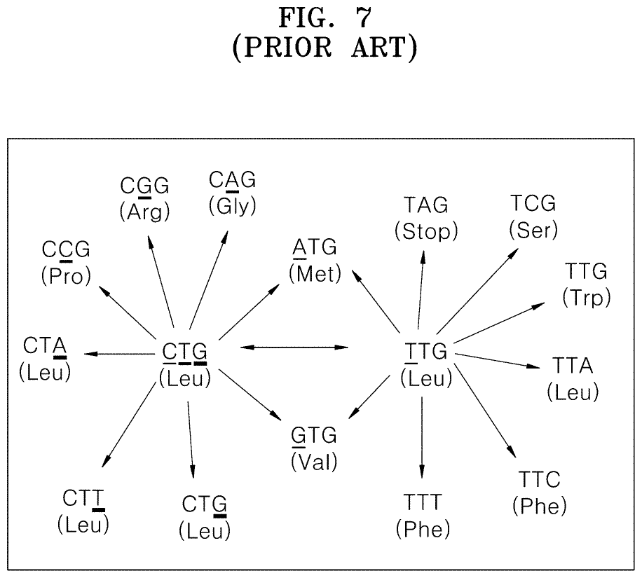

FIG. 7 is an example representation for degeneracy in codons coding for Leucine (CTG, CTA, CTT, CTC, TTG, TTA) and the single nucleotide substitutions at various positions that lead to coding for different amino acids [CTG→CCG (PRO), CCG (Arg), CAG (Gly) changes in middle position], Changes in first position [CTG (Leu)→ATG (Met), GTG (Val)], according to prior art.

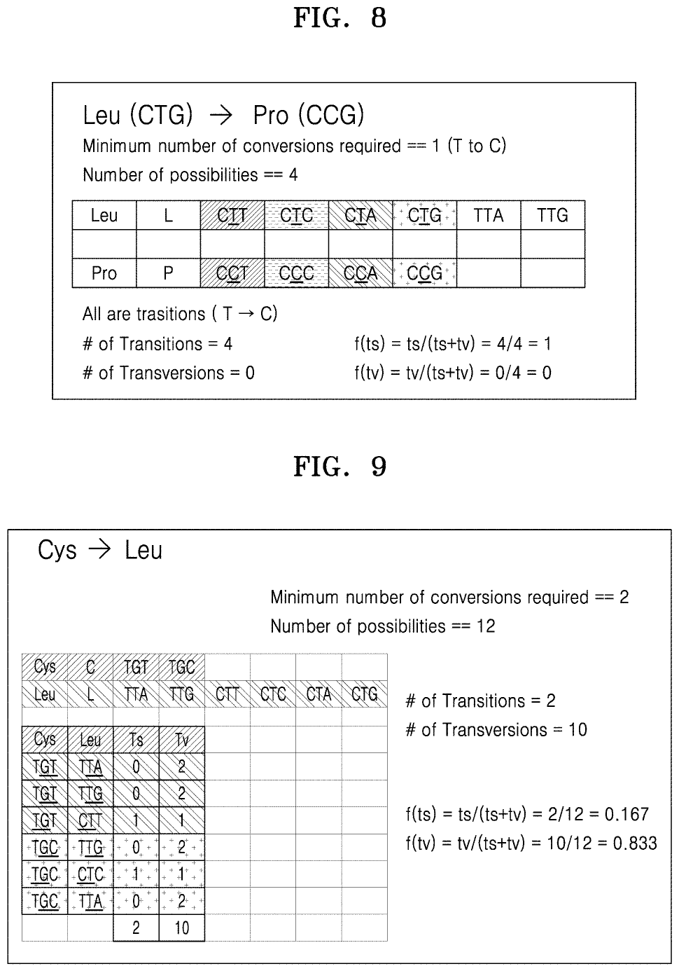

FIG. 8 illustrates an example of nucleotide substitutions that can cause amino acid change from Leucine to Proline, according to an embodiment as disclosed herein. In total there are 4 transitions that can cause this change and there are zero transversions that can cause this change. Based on this information f(ts) and f(tv) are computed as given in formulae.

FIG. 9 illustrates an example of nucleotide substitutions that can cause amino acid change from Cyctein to Leucine, according to an embodiment as disclosed herein. There are a total of 12 possibilities that can make this change of which 2 are transitions and 10 are transversions. Based on this information f(ts)is 0.167 and f(tv) is 0.833.

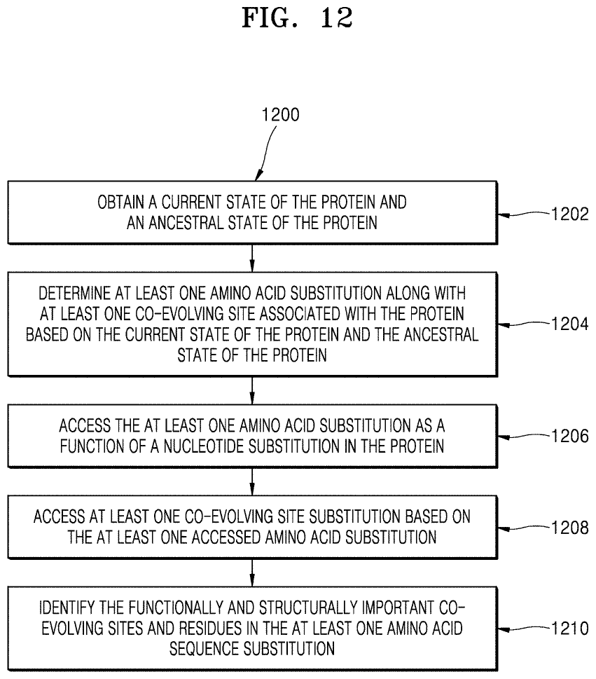

FIG. 12 is a flow chart (1200) illustrating a method for identifying functionally and structurally important co-evolving sites and at least one substituent amino acid residue, according to an embodiment as disclosed herein. The operations (1202-1210) are performed by the processor (110).

At 1202, the method includes obtaining the current state of the protein and the ancestral state of the protein. At 1204, the method includes determining at least one amino acid substitution along with at least one co-evolving site associated with the protein based on the current state of the protein and the ancestral state of the protein. At 1206, the method includes assessing the at least one amino acid substitution as the function of the nucleotide substitution in the protein. At 1208, the method includes assessing at least one co-evolving site substitution based on the at least one assessed amino acid substitution. At 1210, the method includes identifying the functionally and structurally important co-evolving sites and residues in the at least one amino acid sequence substitution.

The various actions, acts, blocks, steps, or the like in the flow chart (1200) may be performed in the order presented, in a different order or simultaneously. Further, in some embodiments, some of the actions, acts, blocks, steps, or the like may be omitted, added, modified, skipped, or the like without departing from the scope of the invention.

The embodiments disclosed herein can be implemented using at least one software program running on at least one hardware device and performing network management functions to control the elements.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

What is claimed is:

1. A method for identifying co-evolving sites, comprising:
obtaining, by an electronic device, a current state of a protein and an ancestral state of the protein;
determining, by the electronic device, at least one amino acid substitution along with at least one co-evolving site associated with the protein based on the current state of the protein and the ancestral state of the protein;
assessing, by the electronic device, the at least one amino acid substitution as a function of a nucleotide substitution in the protein;
assessing, by the electronic device, at least one co-evolving site substitution based on the at least one assessed amino acid substitution; and
identifying, by the electronic device, the at least one co-evolving site based on the at least one assessed co-evolving site substitution.

2. The method as claimed in claim 1, further comprising identifying, by the electronic device, at least one substituent amino acid residue based on the at least one assessed co-evolving site substitution.

3. The method as claimed in claim 1, further comprising ranking, by the electronic device, one of: the co-evolving sites and at least one substituent amino acid residue based on the at least one assessed co-evolving site substitution.

4. The method as claimed in claim 1 or claim 3, further comprising ranking, by the electronic device, co-evolved site pairs based on the co-evolving sites.

5. The method of claim 1, further comprising assessing, by the electronic device, another amino acid substitution based on the co-evolving sites.

6. The method as claimed in claim 1, wherein the at least one co-evolving site substitution is assessed by a type of nucleotide mutation resulting in co-evolving site substitution and assessing a selection pressure on each site.

7. The method as claimed in claim 1, wherein the at least one amino acid substitution is assessed as the function of the nucleotide substitution in the protein using an amino acid substitution matrix, wherein the amino acid substitution matrix reflects a normalized score of count and type of nucleotide substitutions causing amino acid shift.

8. The method as claimed in claim 3, wherein the at least one co-evolving site is ranked based on a weighted average rank of type of nucleotide substitution in the protein and a percentage of at least one amino acid sequence with positional pair.

9. The method as claimed in claim 1, wherein the assessing, by the electronic device, the at least one amino acid substitution as the function of the nucleotide substitution in the protein comprises:
grouping the nucleotide substitution based on a nucleotide transition and a transversion propensity, wherein the nucleotide transition includes substitutions within purines and transversion propensity includes substitutions within pyrimidines; and
assessing the at least one amino acid substitution as the function of the nucleotide substitution in the protein based on the nucleotide transition and the transversion propensity.

10. The method of claim 1, wherein the obtaining the current state of the protein and the ancestral state of the protein comprises:
compiling sequence homologs across taxa;
filtering the sequence homologs;
building multiple sequence alignments; and
obtaining the current state of the protein and the ancestral state of the protein based on the multiple sequence alignments.

11. An electronic device for identifying co-evolving sites and at least one substituent amino acid residue, comprising:
a memory storing instructions;
a processor, coupled with the memory, wherein upon execution of the instructions by the processor the processor is configured to:
obtain a current state of the protein and an ancestral state of the protein;
determine at least one amino acid substitution along with at least one co-evolving site associated with the protein based on the current state of the protein and the ancestral state of the protein;
assess the at least one amino acid substitution as a function of a nucleotide substitution in the protein;
assess at least one co-evolving site substitution based on the at least one assessed amino acid substitution; and
identify the co-evolving sites and at least one substituent amino acid residue based on the at least one assessed co-evolving site substitution.

12. The electronic device as claimed in claim 11, wherein the processor is configured to identify the at least one substituent amino acid residue based on the at least one assessed co-evolving site substitution.

13. The electronic device as claimed in claim 11, wherein the processor is configured to rank one of: the at least one co-evolving site and the at least one substituent amino acid residue based on a nucleotide transition and a transversion propensity.

14. The electronic device as claimed in claim 11, wherein the processor is configured to rank co-evolved site pairs based on the co-evolving sites.

15. The electronic device as claimed in claim 11, wherein the processor is configured to assess another amino acid substitution based on the at least one co-evolving site.

16. The electronic device as claimed in claim 11, wherein the at least one co-evolving site substitution is assessed by a type of nucleotide mutation resulting in co-evolving site substitution and assessing a selection pressure on each site.

17. The electronic device as claimed in claim 11, wherein the at least one amino acid substitution is assessed as the function of the nucleotide substitution in the protein using an amino acid substitution matrix, wherein the amino acid substitution matrix reflects a normalized score of count and type of nucleotide substitutions causing amino acid shift.

18. The electronic device as claimed in claim 13, wherein the at least one site is ranked based on a weighted average rank of type of nucleotide substitution in the protein and a percentage of the at least one amino acid sequence with positional pair.

19. The electronic device as claimed in claim 11, wherein the processor being configured to assess, by the electronic device, the at least one amino acid substitution as the function of the nucleotide substitution in the protein comprises the processor being configured to:
group the nucleotide substitution based on a nucleotide transition and a transversion propensity, wherein the nucleotide transition includes substitutions within purines and the transversion propensity includes substitutions within pyrimidines; and
assess the at least one amino acid substitution as the function of the nucleotide substitution in the protein based on the nucleotide transition and the transversion propensity.

20. The electronic device as claimed in claim 11, wherein the processor being configured to obtain the current state of the protein and the ancestral state of the protein comprises the processor being configured to:
compile sequence homologs across taxa;
filter the sequence homologs;
build multiple sequence alignments; and
obtain the current state of the protein and the ancestral state of the protein based on the multiple sequence alignments.
